(19) United States
(12) Patent Application Publication
(10) Pub. No.: US 2012/0190957 A1
(43) Pub. Date: Jul. 26, 2012

(54) SYSTEM AND METHOD FOR MONITORING CARDIAC DISEASE

(75) Inventors: Jong Gill, Valencia, CA (US); Cecilia Qin Xi, San Jose, CA (US); Stuart Rosenberg, Castaic, CA (US); Yelena Nabutovsky, Sunnyvale, CA (US); Brian Jeffrey Wenzel, San Jose, CA (US); William Hsu, Thousand Oaks, CA (US)

(73) Assignee: PACESETTER, INC., Sylmar, CA (US)

(21) Appl. No.: 13/010,535

(22) Filed: Jan. 20, 2011

(51) Int. Cl. A61B 5/042; A61B 5/0408; A61B 5/0402

(52) U.S. Cl. 600/374; 600/509; 600/393

(57) ABSTRACT

A method of monitoring progression of cardiac disease includes applying stimulus pulses to the heart and sensing electrophysiological responses of the heart at a plurality of different monitoring locations of the heart. The method also includes comparing a previously and subsequently sensed electrophysiological responses that are sensed near a first location of the monitoring locations and comparing previously and subsequently sensed electrophysiological responses that are sensed near a second location of the monitoring locations. The method further includes identifying a change in progression of cardiac disease of the heart based on a difference between the previously and subsequently sensed electrophysiological responses at the first location and based on a difference between the previously and subsequently sensed electrophysiological responses at the second location.

### FIELD OF THE INVENTION

Embodiments described herein generally relate to implantable and external medical devices, and more particularly pertain to methods and systems that monitor cardiac signals to detect cardiac instability, such as cardiac disease, and progression of cardiac disease.

### BACKGROUND OF THE INVENTION

Medical devices are implanted in patients to monitor, among other things, electrical activity of a heart and to deliver appropriate electrical and/or drug therapy, as required. Implantable medical devices (IMDs) include, for example, pacemakers, cardioverters, defibrillators, implantable cardioverter defibrillators (ICD), and the like. The electrical therapy produced by an IMD may include, for example, pacing pulses, cardioverting pulses, and/or defibrillator pulses to reverse arrhythmias (for example, tachycardias and bradycardias) or to stimulate the contraction of cardiac tissue (for example, cardiac pacing) to return the heart to its normal sinus rhythm.

IMDs can deliver stimulus pulses to the heart and measure subsequent electrophysiological responses of the heart to the stimulus pulses, such as evoked responses of the heart. The evoked responses are cardiac signals that reflect contraction or other activity of the heart in response to the stimulus pulses. Some known IMDs measure evoked responses of the heart to detect heart failure (HF), such as by detecting acute decompensated HF (ADHF) events. The IMDs attempt to detect the ADHF events early enough for the user to take action and thereby avoid costly hospitalization associated with the events.

Some known IMDs are limited to detecting HF events, and do not provide information regarding the progression of cardiac disease, such as the progression of HF events. For instance, once patients experience an initial HF event, it is likely that patients will experience additional HF events which lead to the worsening of HF. Moreover, some known IMDs do not provide information on the improving progression, or the improvement, of cardiac disease, in response to therapy, such as cardiac resynchronization therapy and/or medication. Therefore, physicians may be unaware of the efficacy of currently applied treatment to prevent the adverse progression of cardiac disease.

A need exists for systems and methods that provide information on the progression of cardiac disease, such as the positive or adverse progression of HF.

### BRIEF SUMMARY OF THE INVENTION

In one embodiment, a method of monitoring progression of cardiac disease is provided. The method includes applying stimulus pulses to the heart and sensing cardiac signals of the heart at a plurality of different monitoring locations of the heart. The cardiac signals are representative of electrophysiological responses of the heart to the stimulus pulses. The method also includes comparing a previously sensed electrophysiological response that is sensed near a first location of the monitoring locations with a subsequently sensed electrophysiological response that is sensed near the first location. The method further includes comparing a previously sensed electrophysiological response that is sensed near a second location of the monitoring locations with a subsequently sensed electrophysiological response that is sensed near the second location. Additionally, the method includes identifying a change in progression of cardiac disease of the heart based on a difference between the previously and subsequently sensed electrophysiological responses at the first location and based on a difference between the previously and subsequently sensed electrophysiological responses at the second location. In one aspect, the method may include sensing the electrophysiological responses at more than the second location, such as at third, fourth, fifth, and the like, locations.

In another embodiment, a cardiac monitoring system is provided. The system includes an implantable medical device, a monitoring module, and a diagnostic module. The implantable medical device is configured to deliver stimulus pulses to a heart and includes electrodes configured to sense cardiac signals representative of electrophysiological responses of the heart to the stimulation pulses at a plurality of different monitoring locations of the heart. The monitoring module is configured to compare a previously sensed electrophysiological response that is sensed near a first location of the monitoring locations with a subsequently sensed electrophysiological response that is sensed near the first location. The monitoring module also is configured to compare a previously sensed electrophysiological response that is sensed near a second location of the monitoring locations with a subsequently sensed electrophysiological response that is sensed near the second location. The diagnostic module is configured to identify a change in progression of cardiac disease of the heart based on a difference between the previously and subsequently sensed electrophysiological responses obtained near the first location and based on a difference between the previously and subsequently sensed electrophysiological responses obtained near the second location.

In another embodiment, a tangible and non-transitory computer readable storage medium for use in a cardiac monitoring system including a processor and an implantable medical device is provided. The computer readable storage medium includes instructions to direct the processor to determine electrophysiological responses of the heart to stimulus pulses applied to the heart at a plurality of different monitoring locations of the heart. The instructions also direct the processor to compare a previously sensed electrophysiological response obtained near a first location of the monitoring locations with a subsequently sensed electrophysiological response obtained near the first location and to compare a previously sensed electrophysiological response obtained near a second location of the monitoring locations with a subsequently sensed electrophysiological response obtained near the second location. The instructions further direct the processor to identify a change in progression of cardiac disease based on a difference between the previously and subsequently sensed electrophysiological responses obtained near the first location and based on a difference between the previously and subsequently sensed electrophysiological responses obtained near the second location.

### BRIEF DESCRIPTION OF THE DRAWINGS

The drawings illustrate generally, by way of example, but not by way of limitation, various embodiments discussed in the present document.

FIG. 1 illustrates one embodiment of a cardiac instability monitoring system.

FIG. 2 is an illustration of the implantable medical device shown in FIG. 1.

FIG. 3 illustrates an example of an intra-cardiac electrogram (IEGM).

FIG. 4 is a flowchart of a method for monitoring cardiac disease in accordance with one embodiment.

FIG. 5 illustrates several electrophysiological responses that can be obtained in connection with the method shown in FIG. 4.

FIG. 6 illustrates examples of morphology parameter tables that are populated with the baseline morphology parameters calculated in connection with the method shown in FIG. 4.

FIG. 7 illustrates examples of probability curves that may be used in conjunction with the method shown in FIG. 4.

FIG. 8 illustrates a block diagram of examples of internal components of the IMD shown in FIG. 1.

FIG. 9 illustrates a functional block diagram of an example of an external device shown in FIG. 1.

FIG. 10 illustrates a distributed processing system in accordance with one embodiment.

### DETAILED DESCRIPTION OF THE INVENTION

In the following detailed description, reference is made to the accompanying drawings which form a part hereof, and in which are shown by way of illustration specific embodiments in which the described subject matter may be practiced. These embodiments, which are also referred to herein as “examples,” are described in sufficient detail to enable those skilled in the art to practice the claimed subject matter. It is to be understood that the embodiments may be combined or that other embodiments may be utilized, and that structural, logical, and electrical variations may be made without departing from the scope of the disclosed subject matter. For example, embodiments may be used with a pacemaker, a cardioverter, a defibrillator, and the like. The following detailed description is, therefore, not to be taken in a limiting sense, and the scope of the claimed subject matter is defined by the appended claims and their equivalents. In this document, the terms “a” or “an” are used, as is common in patent documents, to include one or more than one. In this document, the term “or” is used to refer to a nonexclusive or, unless otherwise indicated.

In accordance with certain embodiments, multiple electrophysiological responses of a heart are monitored over a period of time to track the progression of cardiac disease, such as heart failure (HF). The electrophysiological responses represent cardiac signals of the heart that are sensed following application of one or more stimulus pulses to different locations of the heart, such as evoked responses of the heart. The stimulus pulses may be applied and the electrophysiological responses sensed in different monitoring locations of the heart by several electrodes joined to a common lead assembly of an implantable medical device (IMD). Information obtained from the electrophysiological responses at the different monitoring locations of the heart may be used to produce cardiac disease progression data that is stored in the IMD. The progression data can be downloaded from the IMD via (1) a programmer during a clinic follow-up visit, (2) a patient care network such as the Merlin.net® network or (3) a programmer in an emergency room (for example, when a patient checks in after experiencing chest pain). As another example, the progression data may be used to prompt the patient to go to the emergency room.

The progression data is used to analyze progression of cardiac disease, such as a worsening or improvement of HF. The progression data may be related to or tracked based on one or more markers, such as hemodynamic performance, cardiac output, systolic or diastolic blood pressure, contractility, stroke volume, systolic time, Q-wave to onset of systole time, QRS to onset of systole time, and the like. The term “progression” of cardiac disease is intended to include both the improvement of cardiac disease and the worsening of cardiac disease. The progression data may be used to change cardiac therapy of the patient, such as by altering one or more parameters of the therapy. By way of example only, one or more parameters of cardiac resynchronization therapy (CRT) may be adjusted, such as the AV interval, the VV timing, and/or the LV lead location of the CRT.

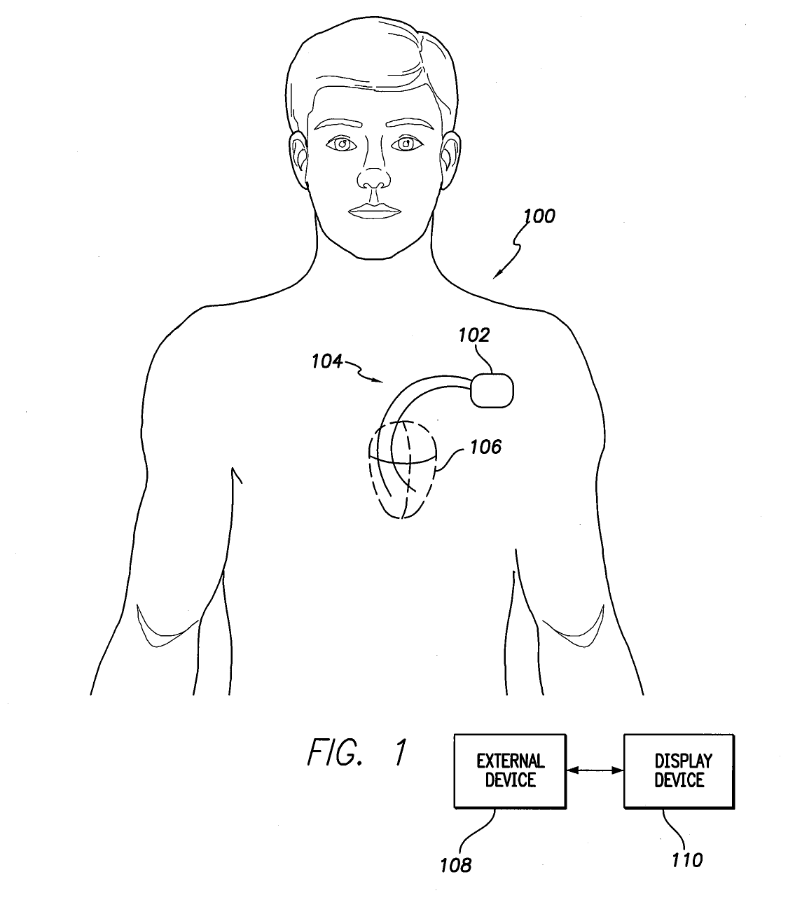

FIG. 1 illustrates a cardiac instability monitoring system 100. The system 100 includes an implantable medical device (IMD) 102 and an external device 108. The IMD 102 may include a cardiac stimulation device that incorporates internal components for controlling HF evaluation functions described below. For example, the IMD 102 may be a cardiac pacemaker, an ICD, a defibrillator, an ICD coupled with a pacemaker, a CRT pacemaker, a cardiac resynchronization therapy defibrillator (CRT-D), and the like. The IMD 102 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. Alternatively, the IMD 102 may be a triple- or quad-chamber stimulation device. Optionally, the IMD 102 may be a multisite stimulation device capable of applying stimulation pulses to multiple sites within each of one or more chambers of a heart 106.

The IMD 102 delivers stimulus pulses to the heart 106 through one or more lead assemblies 104 implanted within the heart 106. The IMD 102 also senses cardiac signals of the heart 106 using the lead assemblies 104. The IMD 102 monitors electrophysiologic responses of the heart 106 to application of the stimulus pulses, such as by sensing the evoked responses of the heart 106 to the stimulus pulses. The IMD 102 senses the electrophysiological responses at a plurality of monitoring locations of the heart 106.

The electrophysiologic responses represent conducted responses, conducted activation, or far-field activation of the heart 106 to stimulation pulses. For example, the IMD 102 may deliver stimulus pulses to the heart 106 and sense cardiac signals of the heart 106 at different monitoring locations that are spaced apart from each other. The cardiac signals sensed at the different monitoring locations represent the localized electrophysiological responses of the heart 106 at or near the different monitoring locations. The waveform morphology of each individual electrophysiological response can provide information regarding the cardiac tissue that is relatively local to the location of the electrode used to obtain the corresponding electrophysiological response.

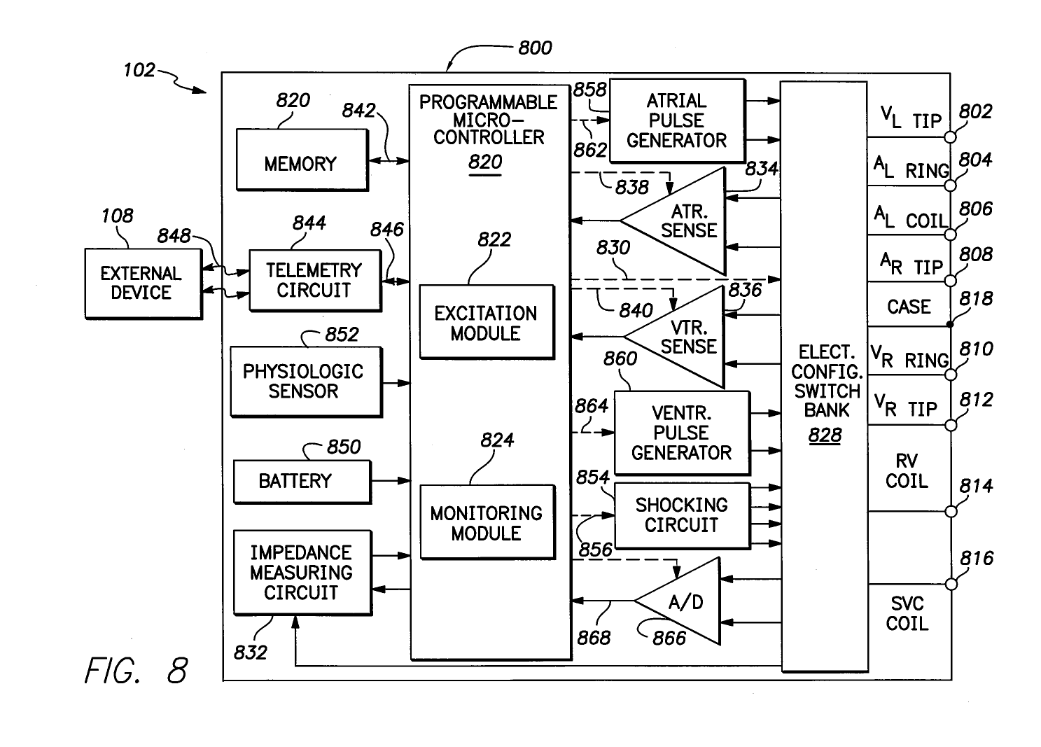

Several electrophysiological responses are obtained at the same monitoring locations over a period of time. The IMD 102 determines the cardiac disease progression data from the electrophysiological responses. The progression data can represent differences in the electrophysiological responses obtained at the same monitoring location at different times. For example, the progression data may represent a difference between a baseline value calculated at a previous time and a morphology parameter of an electrophysiological response sensed at a subsequent time. The electrophysiological responses and/or progression data can be stored in the IMD 102, such as in a memory 826 (shown in FIG. 8) of the IMD 102.

The external device 108 is a device that receives the progression data from the IMD 102. For example, the external device 108 may be a programmer or other computer processor-based device that wirelessly receives the progression data from the IMD 102. Alternatively, the external device 108 receives the electrophysiological responses from the IMD 102 and calculates the progression data. The external device 108 examines the progression data to track or monitor changes in cardiac disease of the heart 106.

The external device 108 can notify an operator of the system 100, such as the patient or a physician, of changes in the progression of cardiac disease. The external device 108 may be coupled with a display device 110, such as a monitor, that visually presents progression data, recommended changes to CRT, drug therapy, or lifestyle modifications based on the progression data, electrophysiological responses, baseline values, thresholds, and the like. The operator can utilize this information to begin, change, or stop providing therapy to the patient. For example, if cardiac instability is discovered, the operator may begin CRT with the patient. If the cardiac instability is negatively progressing, then the operator may determine that one or more parameters of the CRT provided to the patient need to be changed. If the cardiac instability is positively progressing, then the operator may decide to change parameters of the CRT or stop the CRT.

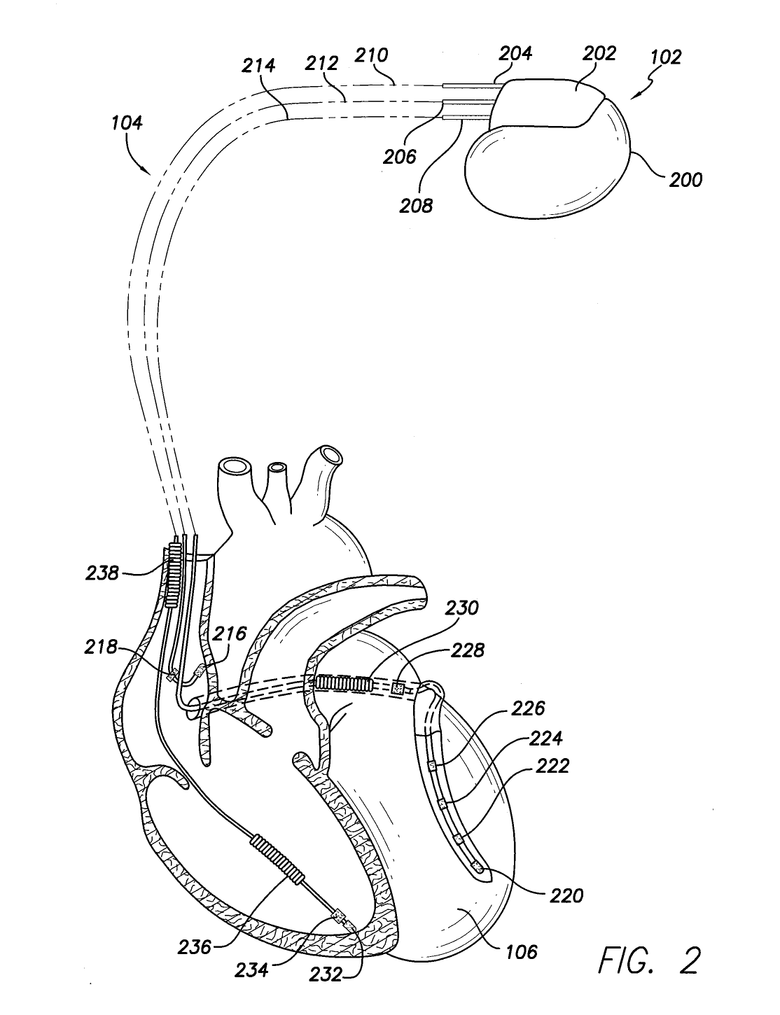

FIG. 2 is an illustration of the IMD 102 that is coupled to the heart 106. The IMD 102 includes a housing 200 joined to a header assembly 202 that holds receptacle connectors 204, 206, 208. The receptacle connectors 204, 206, 208 are connected to the lead assemblies 104. The lead assemblies 104 include a right ventricular (RV) lead 210, a right atrial (RA) lead 212, and a coronary sinus lead 214. The leads 210, 212, 214 are located within the heart 106 to deliver stimulus pulses to the heart 106 and to measure physiological parameters of the heart 106, such as electrophysiological responses of the heart 106 to the stimulus pulses. One or more of the leads 210, 212, 214 may detect IEGM signals that form an electrical activity indicator of myocardial function over multiple cardiac cycles.

The IMD 102 includes several electrodes joined to the leads 210, 212, 214 that are used to deliver the stimulus pulses and/or to sense cardiac signals representative of the electrophysiological responses. The housing 200 of the IMD 102 may be one of the electrodes and is often referred to as the “can”, “case”, or “case electrode.” The RA lead 212 includes an atrial tip electrode 216 and an atrial ring electrode 218. The coronary sinus lead 214 receives atrial and ventricular cardiac signals and delivers left ventricular pacing therapy using at least a left ventricular (LV) tip electrode 220. Optionally, the coronary sinus lead 214 may deliver left atrial (LA) pacing therapy using at least a left atrial ring electrode 228. In one embodiment, the coronary sinus lead 214 delivers shocking therapy using an LA coil electrode 230. The coronary sinus lead 214 may be a quadripolar lead having a plurality of LV electrodes 222, 224, 226, such as ring electrodes, disposed at different locations within the left ventricle between the LV tip electrode 220 and the LA ring electrode 228. The right ventricular (RV) lead 210 is coupled with an RV tip electrode 232, the RV ring electrode 234, and the RV coil electrode 236. The RV lead 210 may include an SVC coil electrode 238. The RV lead 210 is capable of delivering stimulation in the form of pacing and shock therapy to the right ventricle.

As shown in FIG. 2, the electrodes are disposed at different locations of the heart 106 along the respective leads 210, 212, 214. The electrodes can be referred to as “distal” and “proximal” electrodes relative to other electrodes along a common lead 210, 212, 214 or within the same chamber of the heart 106. By way of example only, the LV tip electrode 220 may be referred to as a distal electrode of the lead 214 and the LV ring electrodes 222, 224, 226 referred to as proximal electrodes of the lead 214 relative to the LV tip electrode 220.

The number of electrodes joined to each lead 210, 212, 214 is merely an example. A different number of electrodes may be provided than what is shown in FIG. 2. For example, while the lead 214 is shown as a quadripolar lead having four electrodes in the left ventricle, alternatively, the lead 214 may have a different number of electrodes in the left ventricle.

Alternatively, one or more of the electrodes that delivers stimulus pulses and/or senses cardiac signals may be decoupled from the leads 210, 212, 214. For example, one or more electrodes may be a leadless electrode having a radio frequency (RF) receiver that wirelessly receives energy from the IMD 102 and delivers at least some of the energy as a stimulus pulse. The leadless electrode may include an RF transmitter that transmits cardiac signals sensed by the leadless electrode to the IMD 102.

The IMD 102 applies stimulus pulses to the heart 106 and measures localized electrophysiologic responses of the heart 106 to the stimulus pulses. The stimulus pulses may be stimulus pulses delivered to one or more chambers of the heart 106. A stimulus pulse may be a non-regular stimulus pulse provided outside or in place of a regularly timed or periodic pacing pulse. For example, the stimulus pulse may be a non-therapeutic application of a stimulus pulse that is not related to adjusting or maintaining a rhythm of the cardiac cycles of the heart 106.

The stimulus pulses are applied to the left ventricle of the heart 106 in one embodiment. For example, the one or more stimulus pulses may be delivered to the heart 106 using the LV tip electrode 220 and the LV ring electrodes 222, 224, 226. Cardiac signals are sensed by several electrodes located at the different monitoring locations of the heart 106. The monitoring locations represent the position of the sensing electrode within or near the heart 106. In the illustrated embodiment, the LV tip electrode 220 and each of the LV ring electrodes 222, 224, 226 sense cardiac signals representative of electrophysiological responses to the stimulus pulse.

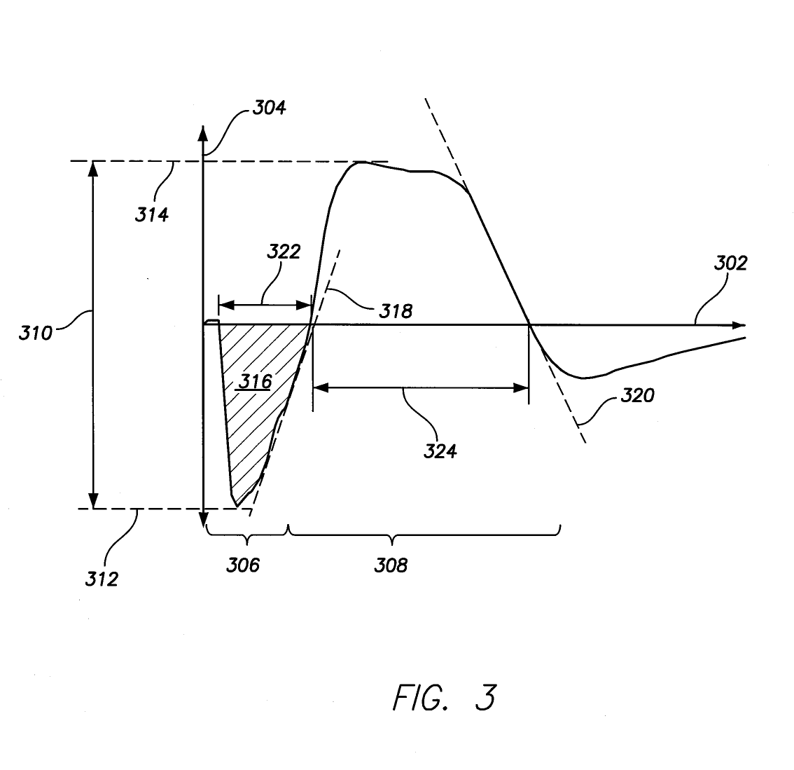

FIG. 3 illustrates an intra-cardiac electrogram (IEGM) 300 representative of cardiac signals of the heart 106 (shown in FIG. 1). The IEGM 300 represents an electrophysiological response of the heart 106 that is measured by one or more of the electrodes disposed within the heart 106. The IEGM 300 is shown on a graph having a horizontal axis 302 and a vertical axis 304. The horizontal axis 302 represents time and the vertical axis 304 represents the voltage of the cardiac signals measured by the IMD 102 (shown in FIG. 1) using one or more of the electrodes.

The IEGM 300 includes cardiac signal waveform segments comprised of an R-wave 306 and a T-wave 308. The R- and T-waves 306, 308 represent the behavior of the ventricles of the heart 106 (shown in FIG. 1). Certain waveform morphology parameters of the IEGM 300 may be measured to allow the electrophysiological response represented by the IEGM 300 to be compared to another electrophysiological response sensed at the same monitoring location at a different time. The morphology parameters include characterizations of one or more waveform segments of the IEGM 300 that quantify the IEGM 300.

A peak-to-peak height index 310 is one morphology parameter that can be calculated for the IEGM 300. In the illustrated embodiment, the peak-to-peak height index 310 represents the difference between a negative peak value 312 and a positive peak value 314 of the IEGM 300. The peak-to-peak height index 310 can be calculated as the sum of the absolute values of the amplitudes of the R-wave 306 and the T-wave 308, with the negative peak value 312 representing the amplitude of the R-wave 306 and the positive peak value 314 representing the amplitude of the T-wave 308. In one embodiment, one or more of the peak values 312, 314 may be morphology parameters. In a patient suffering from pulmonary edema having a cardiac origin, the peak-to-peak height index 310 may vary among several evoked responses. For example, the peak-to-peak height index 310 may increase or decrease between different electrophysiological responses obtained at different times but at the same monitoring location.

A paced depolarization integral (PDI) 316 is another morphology parameter that can be examined for the IEGM 300. The PDI 316 represents the area between the R-wave 306 and a baseline value of the IEGM 300. The baseline value of the IEGM 300 is the relatively steady value of the IEGM 300 prior to applying the stimulus pulse to the heart 106 (shown in FIG. 1). After applying the stimulation pulse, the IEGM 300 decreases to form the R-wave 306. The IEGM 300 may return to the baseline value before increasing above the baseline value to form the T-wave 308. In the illustrated embodiment, the baseline value is equal to the horizontal axis 302. For example, the baseline value may be zero volts. Alternatively, the baseline value may be disposed above or below the horizontal axis 302.

The PDI 316 is determined by calculating the area between the R-wave 306 and the baseline value. For example, the PDI 1012 may be calculated by integrating under the R-wave 306. In a patient suffering from cardiac disease, the PDI 306 may vary among several evoked responses. For example, the size and/or shape of the R-wave 306 may be different among electrophysiological responses that are sensed at different times but at the same monitoring location. As the R-waves 306 change, the PDI 306 of the electrophysiological responses also may change and be indicative of cardiac disease and/or progression of cardiac disease.

Slope indices 318, 320 are additional morphology parameters that can be examined for the IEGM 300. The slope indices 318, 320 represent changes in amplitude of the IEGM 300 divided by the corresponding change in time for a segment of the IEGM 300. In the illustrated embodiment, the slope indices 318, 320 include a peak slope index 318 and a non-peak slope index 320 is a straight line that is fit to a portion of the R-wave 306 shortly after the negative peak value 312. For example, the peak slope index 318 may be fit to the portion of the R-wave 306 that extends from the negative peak value 312 or shortly after the negative peak value 312 to the baseline value. The non-peak slope index 320 is a straight line that is fit to another portion of the IEGM 300. In the illustrated embodiment, the non-peak slope index 320 is fit to the decreasing portion of the T-wave 308. Alternatively, the slope indices 318, 320 may represent the slopes of the IEGM 300 at one or more other locations.

One or more of the slope indices 318, 320 can vary among several evoked responses for a patient suffering from cardiac disease. For example, the morphology of the R-wave 306 may change over time due to the progression or onset of cardiac disease. As the R-wave 306 morphology changes, the slope indices 318 and/or 320 also may change between electrophysiological responses that are obtained at different times but at the same monitoring location. These changes can indicate onset or progression of cardiac disease, such as HF.

Segment width indices 322, 324 are additional morphology parameters that can be examined for the IEGM 300. The width indices 322, 324 represent time periods over which each of the R-wave 306 and the T-wave 308 extend, respectively. For example, the width index 322 may represent the time period over which the R-wave 306 decreases from the baseline value to the negative peak value 312 and then increases from the negative peak value 312 to the baseline value. The width index 324 may represent the time period over which the T-wave 308 increases from the baseline value to the positive peak value 314 and then decreases from the positive peak value 314 to the baseline value. The morphology of the R-wave 306 and/or T-wave 308 may change over time due to the progression or onset of cardiac disease. As the morphology of the R-wave 306 and/or T-wave 308 changes, the width indices 322, 324 also may change between electrophysiological responses that are obtained at different times but at the same monitoring location. These changes can indicate onset or progression of cardiac disease, such as HF.

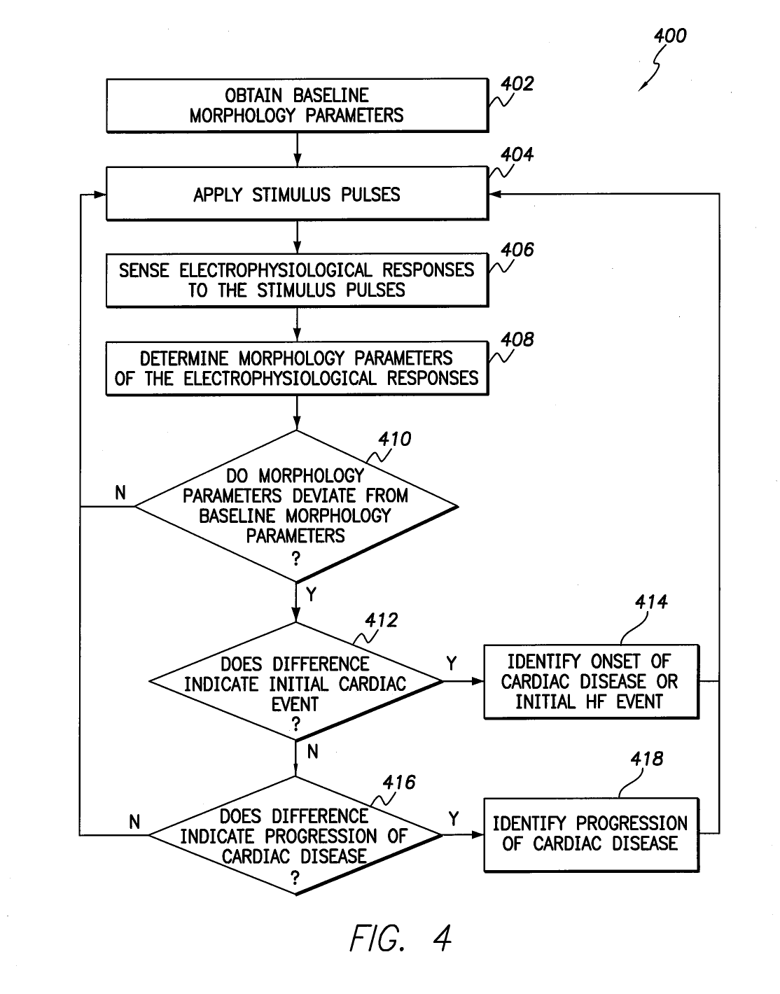

FIG. 4 is a flowchart of a method 400 for monitoring cardiac disease in accordance with one embodiment. At 402, baseline morphology parameters are obtained for electrophysiological responses sensed at a plurality of monitoring locations. The baseline morphology parameters may be obtained prior to onset of cardiac disease, or prior to detection of an initial HF event. For example, the baseline morphology parameters may be calculated before the cardiac output of a patient decreases below a threshold. In one embodiment, the baseline morphology parameters are obtained when the patient is in a sedentary or non-active state. For example, the baseline morphology parameters may be obtained during a time period when the patient is resting, or is not moving or exercising (such as walking, jogging, or running).

In one embodiment, the baseline morphology parameters are determined by delivering stimulus pulses to the heart 106 (shown in FIG. 1). The electrophysiological responses of the heart 106 to the stimulus pulses are sensed by several electrodes at or near a plurality of different monitoring locations of the heart 106. For example, the electrodes 220, 222, 224, 226 (shown in FIG. 2) disposed at various spaced apart positions in the left ventricle of the heart 106 can sense the electrophysiological responses to the stimulus pulses. Alternatively, different electrodes may be used.

In another embodiment, the baseline morphology parameters may be manually determined by an operator. For example, a physician can select the baseline morphology parameters and transmit the parameters to the IMD 102 (shown in FIG. 1) via the external device 108 (shown in FIG. 1).

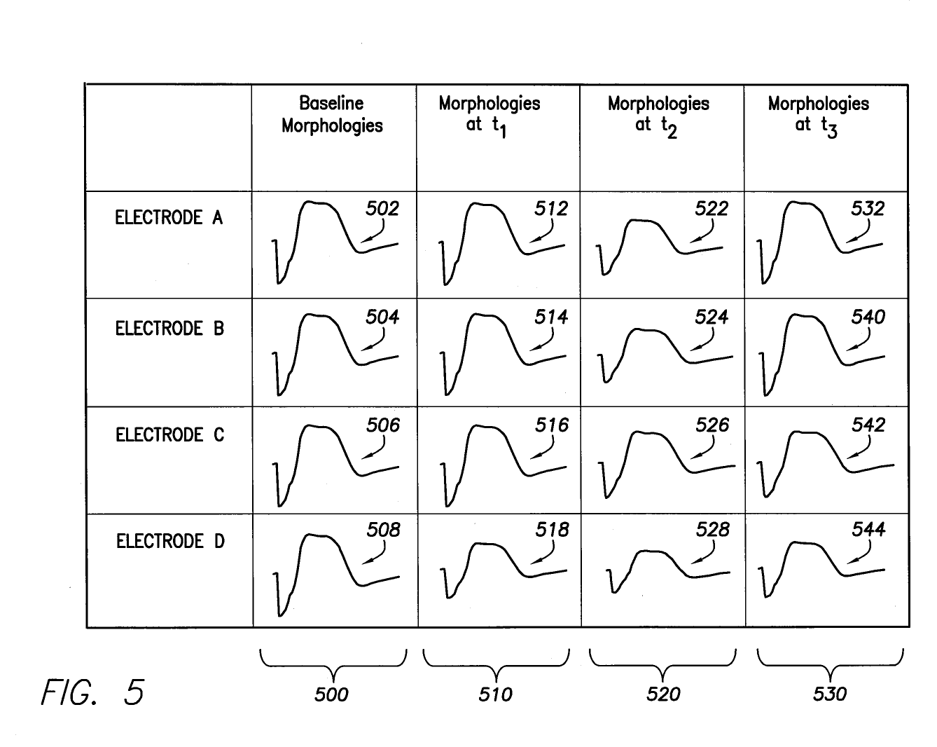

FIG. 5 illustrates several electrophysiological responses that can be obtained in connection with the method 400 shown in FIG. 4. A baseline set 500 of electrophysiological responses 502, 504, 506, 508 represents the cardiac signals sensed by the electrodes 220, 222, 224, 226 (shown in FIG. 2) to establish the baseline morphology parameters. The electrophysiological responses 502, 504, 506, 508 of the baseline set 500 may be referred to as baseline electrophysiological responses.

In the illustrated embodiment, the electrophysiological response 502 is sensed by the electrode 226 (referred to as “Electrode A”), the electrophysiological response 504 is sensed by the electrode 224 (referred to as “Electrode B”), the electrophysiological response 506 is sensed by the electrode 222 (referred to as “Electrode C”), and the electrophysiological response 508 is sensed by the electrode 220 (referred to as “Electrode D”). As shown in FIG. 2, Electrode D is a distal electrode (the LV tip electrode 220) relative to the Electrodes A through C (the LV ring electrodes 226, 224, 222, respectively), which may be referred to as proximal electrodes.

The baseline morphology parameters are calculated for the baseline electrophysiological responses 502, 504, 506, 508. One or more baseline morphology parameters may be calculated for each baseline electrophysiological response 502, 504, 506, 508. For example, one or more of the negative peak value 312, positive peak value 314, the peak-to-peak height index 310, the PDI 316, the width index 322 and/or 324, and/or the slope index 318 and/or 320 (all shown in FIG. 3) may be calculated for each of the baseline electrophysiological responses 502, 504, 506, 508. The baseline morphology parameters are stored in a memory, such as the memory 826 (shown in FIG. 8) of the IMD 102 (shown in FIG. 1).

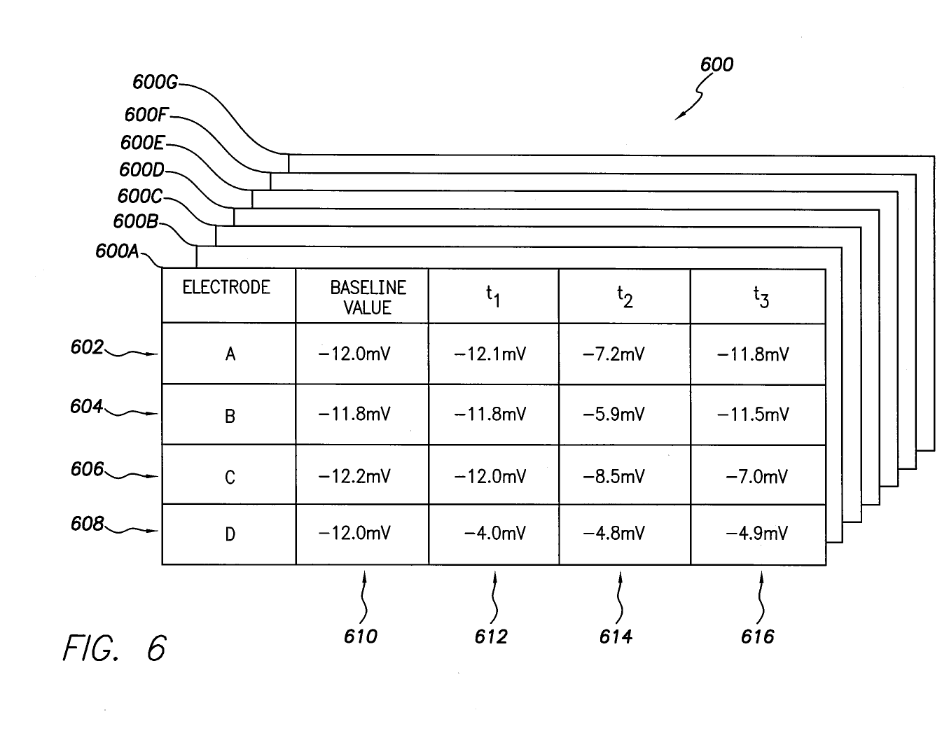

FIG. 6 illustrates examples of morphology parameter tables 600 that are populated with the baseline morphology parameters calculated in connection with the method 400 shown in FIG. 4. Several tables 600A, 600B, 600C, 600D, 600E, 600F, 600G are shown, with each table 600 populated with values of a different morphology parameter. For example, the table 600A may be populated with values of the peak negative value 312 (shown in FIG. 3), table 600B may be populated with the values of the peak-to-peak height index 310 (shown in FIG. 3), and so on.

The tables 600 represent examples of data structures that can be used to record morphology parameters that are calculated based on electrophysiological responses sensed at the monitoring locations of the heart 106 (shown in FIG. 1). The tables 600 record the morphology parameters to enable identification of changes in morphology parameters over time at one or more of the monitoring locations of the heart 106. The progression of cardiac disease may be identified based on the changes in the morphology parameters over time at a plurality of the monitoring locations.

The tables 600 include rows 602, 604, 606, 608 that correspond to the electrodes that sense the electrophysiological responses used to measure the morphology parameter. For example, with respect to the negative peak values 312 (shown in FIG. 3) that are recorded in the table 600A, the rows 602, 604, 606 correspond to the negative peak values 312 measured by the proximal Electrodes A, B, and C, respectively, while the row 608 corresponds to the negative peak values 312 measured by the distal Electrode D.

The tables 600 also include columns 610, 612, 614, 616 that correspond to different times at which the morphology parameters are measured. For example, with respect to table 600A, the column 610 is used to record the negative peak values 312 (shown in FIG. 3) measured for the baseline set 500 (shown in FIG. 5) of electrophysiological responses 502, 504, 506, 508 (shown in FIG. 5). For example, the first column 610 records the morphology parameters measured by the Electrodes A, B, C, and D, while the second through fourth columns 612, 614, 616 record the corresponding morphology parameters measured by the same Electrodes A, B, C, and D at later times.

Returning to the discussion of the method 400 shown in FIG. 4, the baseline morphology parameters are recorded in the first column 610 (shown in FIG. 6) of the tables 600 (shown in FIG. 6). These baseline morphology parameters may be periodically updated. For example, the baseline morphology parameters can be adjusted or recalculated once per day, week, month, or year. Alternatively, the baseline morphology parameters may represent average, median, or other values of several baseline morphology parameters measured at different times. In another embodiment, the baseline morphology parameters may be periodically updated by an operator. For example, a physician may input the baseline morphology parameters using the external device 108 (shown in FIG. 1) at regular or non-regular time intervals.

Once the baseline morphology parameters are established, additional electrophysiological responses are obtained and additional morphology parameters are measured to allow comparisons between the additional morphology parameters and the baseline morphology parameters. These comparisons may yield differences between the baseline and additional morphology parameters measured at one or more monitoring locations of the heart 106 (shown in FIG. 1). The onset or progression of cardiac disease may be quantified based on these differences.

At 404, one or more stimulus pulses are applied to the heart 106 (shown in FIG. 1). The stimulus pulses may be referred to as “subsequent” stimulus pulses because the stimulus pulses are delivered to the heart 106 after the baseline morphology parameters are established. The subsequent stimulus pulses can be delivered to the heart 106 at or near the same locations that the stimulus pulses used to establish the baseline morphology parameters.

At 406, electrophysiological responses to the subsequent stimulus pulses are sensed. The electrophysiological responses may be referred to as subsequent electrophysiological responses. The subsequent electrophysiological responses may be cardiac signals of evoked responses that are sensed by the Electrodes A, B, C, and D. As shown in FIG. 5, the subsequent electrophysiological responses are identified as an updated set 510 of electrophysiological responses 512, 514, 516, 518. The subsequent electrophysiological responses are sensed at the same monitoring locations of the heart 106 (shown in FIG. 1) where the baseline electrophysiological responses were sensed.

In one embodiment, the subsequent electrophysiological responses are sensed during an active or non-sedentary state of the patient. For example, the subsequent electrophysiological responses may be sensed when the patient is walking, jogging, running, or otherwise acting in a non-resting state.

At 408, morphology parameters of the subsequent electrophysiological responses are determined. The morphology parameters are referred to as “subsequent” morphology parameters because the morphology parameters are calculated based on the subsequent electrophysiological responses. The subsequent morphology parameters can be calculated based on the subsequent electrophysiological responses 512, 514, 516, 518 (shown in FIG. 5) of the updated set 510 (shown in FIG. 5).

The subsequent morphology parameters are the same type of morphology parameters as the baseline morphology parameters in one embodiment. For example, if the baseline morphology parameter calculated from the electrophysiological response sensed by Electrode A is a peak-to-peak height index 310 (shown in FIG. 3), then the subsequent morphology parameter calculated for the subsequent electrophysiological response of sensed by Electrode A also is a peak-to-peak height index 310.

The same morphology parameters may be calculated for the subsequent morphology parameters as the baseline morphology parameters each time the subsequent morphology parameters are calculated. For example, if the baseline morphology parameters include the slope index 318 (shown in FIG. 3) and the PDI 316 (shown in FIG. 3) calculated for each monitoring location of the heart 106 (shown in FIG. 1), then the subsequent morphology parameters can include the slope index 318 and PDI 316 for each monitoring location. Alternatively, the subsequent morphology parameters calculated at one or more of the monitoring locations can be a subset of the baseline morphology parameters calculated at the monitoring locations.

The subsequent morphology parameters are stored in a memory, such as the memory 826 (shown in FIG. 8) of the IMD 102 (shown in FIG. 1). Returning to the discussion of the tables 600 shown in FIG. 6, the subsequent morphology parameters can be recorded in the corresponding tables 600. The first set of subsequent morphology parameters calculated after the baseline morphology parameters can be recorded in the second column 612 of the tables 600. The second column 612 is labeled “t₁” because the corresponding morphology parameters were obtained at a first time after the baseline morphology parameters were established, or after a time t₀.

Returning to the discussion of the method 400 shown in FIG. 4, at 410, the subsequent morphology parameters are compared to the baseline morphology parameters to identify changes in the morphology parameters. For example, differences between the subsequent morphology parameters and the baseline morphology parameters at the monitoring locations (or a subset of the monitoring locations) can be calculated. Differences between the subsequent morphology parameters and the baseline morphology parameters may indicate that the subsequent electrophysiological responses deviate from the baseline electrophysiological responses at one or more of the monitoring locations of the heart 106 (shown in FIG. 1). Such deviation can represent an initial cardiac event, such as an initial HF event, and/or the progression of cardiac disease, such as the worsening or improvement of HF.

In one embodiment, the differences between the baseline and subsequent morphology parameters are compared to one or more thresholds to determine if the differences are significant differences. For example, some differences between the morphology parameters may be insignificant in that the differences do not indicate onset or progression of cardiac disease and are caused by other factors. The thresholds may be based on standard deviations or some other statistical analysis of previously acquired morphology parameters. For example, a data set of previously acquired morphology parameters at each of the monitoring locations may be examined to determine a standard deviation characteristic of the data set. The standard deviation characteristics for the different monitoring locations may be used as corresponding thresholds for the monitoring locations. The standard deviation characteristics may be periodically updated as additional morphology parameters are obtained. For example, the standard deviation characteristics may be updated as additional morphology parameters are calculated based on electrophysiological responses sensed during non-cardiac events, such as non-HF events.

Alternatively, the thresholds for the monitoring locations may be predetermined, such as values that are previously stored in the memory 826 (shown in FIG. 8) of the IMD 102 (shown in FIG. 1) and that do not change based on calculated morphology parameters. For example, an operator may manually set the values of the thresholds for one or more of the monitoring locations.

In one embodiment, one or more of the thresholds may be updated or adjusted over time. The thresholds may be adjusted to account for and/or reduce the incidences of false positives and/or false negatives. For example, if a threshold is set to a value that is too high, the differences between the baseline and subsequent morphology parameters may not indicate a worsening of cardiac disease when the cardiac disease is getting worse. Alternatively, if a threshold is set to a value that is too low, the differences between baseline and subsequent morphology parameters may indicate a worsening of cardiac disease when cardiac disease is not getting worse or is improving. In order to adjust the thresholds to reduce such false positives and/or false negatives, other cardiac signals may be examined. For example, during a time period when the differences between baseline and subsequent morphology parameters exceed one or more thresholds and therefore indicate a worsening cardiac disease, other cardiac signals (e.g., morphology parameters of other cardiac signals, time intervals of cardiac waveforms, etc.) may be examined to determine if the other cardiac signals support the finding of a worsening of the cardiac disease. If the other cardiac signals do not support the finding of a worsening cardiac disease, then one or more of the thresholds may be increased for future comparisons with differences between the baseline and subsequent morphology parameters. Increasing the thresholds can require larger differences between the baseline and subsequent morphology parameters in order to identify a worsening or negative progression of cardiac disease.

In another example, the patient may notify the physician of time periods when the patient experiences physical discomfort that may be associated with a cardiac episode that may be indicative of a worsening of cardiac disease. The physician may then compare the current thresholds with the morphology parameters (such as the differences between baseline and subsequent morphology parameters) associated with the time periods of physical discomfort. If the differences do not exceed one or more of the thresholds during these time periods, then the thresholds may be set to too high. For example, the morphology parameters and/or the differences between the morphology parameters may represent cardiac episodes caused by or associated with cardiac disease and/or may represent a worsening of cardiac disease, but the thresholds may be set too high for the morphology parameters to indicate the cardiac episode and/or worsening cardiac disease. As a result, the thresholds may be lowered, such as by a predetermined amount or a manually set amount. The lowered thresholds may be more sensitive to identifying future cardiac episodes and/or worsening of cardiac disease.

The differences between the subsequent and baseline morphology parameters at each monitoring location are compared to the corresponding threshold. If one or more of the differences exceeds an associated threshold, then the differences may indicate an initial event of cardiac instability, such as an initial HF event, and/or progression in cardiac disease, such as a worsening or improvement of HF. As a result, flow of the method 400 proceeds to 412. In one embodiment, flow of the method 400 proceeds to 412 when at least a predetermined number of the differences exceed associated thresholds. In another embodiment, flow of the method 400 proceeds to 412 when at least a predetermined number of different morphology parameters have differences that exceed associated thresholds.

In contrast, if none of the differences exceeds an associated threshold (or if less than a predetermined number of differences does not exceed associated thresholds and/or less than a predetermined number of morphology parameters have differences that do not exceed associated thresholds), then flow of the method 400 may return to 404. For example, the method 400 may return to 404 in a loop-wise manner with additional subsequent physiological parameters being obtained and recorded in the tables 600 (shown in FIG. 6). The additional subsequent morphology parameters can be recorded in the tables 600 in the other columns 614, 616 (shown in FIG. 6). For example, the set of morphology parameters obtained after the morphology parameters recorded in column 612 may be recorded in the column 614 (labeled “t₂”), the following set of morphology parameters may be recorded in the next column 616 (labeled “t₃”), and so on. The additional subsequent morphology parameters recorded in the columns 614, 616, and so on, can be compared to the baseline morphology parameters to repeatedly check on the onset or progression of cardiac disease.

In another embodiment, one or more of the additional subsequent morphology parameters may be compared with previously calculated, non-baseline morphology parameters. This comparison may be used in place of or in addition to the comparison between the baseline and the subsequent morphology parameters described above. For example, the determination of whether significant differences exist between the morphology parameters obtained at one or more monitoring locations may be based on comparisons between the baseline morphology parameters and a first set of subsequent morphology parameters and between the first set and a second set of subsequent morphology parameters.

With respect to the negative peak value morphology parameters recorded in the table 600A shown in FIG. 6, differences between the negative peak values 312 (shown in FIG. 3) measured by each of the Electrodes A, B, C, and D are calculated. In the illustrated embodiment, the absolute values of the differences between the baseline and subsequent negative peak values 312 for the Electrodes A, B, C, and D are shown in Table 1 below.

TABLE 1

| | Baseline | Subsequent | | |
|---|---|---|---|---|
| | morphology | morphology | Absolute | |
| Electrode | parameter | parameter | difference | Threshold |
| A | −12.0 millivolts | −12.1 mV | 0.1 mV | 4.0 mV |
| | (mV) | | | |
| B | −11.8 mV | −11.8 mV | 0.0 mV | 4.0 mV |
| C | −12.2 mV | −12.0 mV | 0.2 mV | 4.0 mV |
| D | −12.0 mV | −4.0 mV | 8.0 mV | 7.0 mV |

The individual thresholds associated with the Electrodes A, B, C, and D also are displayed in the above table. As shown above, the threshold associated with the distal Electrode D may be significantly larger than the thresholds of the proximal Electrodes A, B, and C. The distal Electrode D or another electrode that is closer to the free wall of the left ventricle may have a larger threshold because the free wall may be subject to a greater increase in loading relative to other cardiac tissue of the left ventricle during a cardiac event. The greater increase in loading can result in larger differences in the electrophysiological responses sensed by the distal Electrode D relative to the other Electrodes A, B, and C. As a result, the threshold associated with the distal Electrode D may be greater than the thresholds of the Electrodes A, B, and C. Alternatively, the threshold for Electrode D may be smaller or approximately the same as one or more other thresholds of Electrodes A, B, or C. In another embodiment, a single threshold is used for several or all of the Electrodes A, B, C, and D.

Alternatively, the differences between the baseline and subsequent morphology parameters may be represented by a morphology score. The morphology score indicates a degree or amount of change in the subsequent morphology parameter relative to the baseline morphology parameter. The morphology score may be calculated as a normalized relationship between the baseline and subsequent morphology parameters. For example, the morphology score for a morphology parameter may be calculated using the following

$$M = \frac{{P}_{i}}{{P}_{B}} \qquad ( Equation \#1 )$$

where M represents the morphology score, Pᵢ represents the subsequent morphology parameter, and P_{B} represents the baseline morphology parameter.

In one embodiment, the morphology scores are calculated for amplitude-dependent morphology parameters. For example, the morphology scores may be calculated for the morphology parameters having values that are dependent on an amplitude of the waveform of the electrophysiological response. By way of example only, the morphology scores may be calculated for one or more of the positive peak value 312 (shown in FIG. 3), the negative peak value 314 (shown in FIG. 3), and/or the PDI 316 (shown in FIG. 3). Alternatively, the morphology scores are calculated for amplitude-independent morphology parameters, such as the morphology parameters having values that are not dependent on an amplitude of the waveform. For example, the amplitude-independent morphology scores may be calculated for one or more of the slope indices 318, 320 (shown in FIG. 3) and/or the width indices 322, 324 (shown in FIG. 3).

In the illustrated embodiment, the morphology scores for the Electrodes A, B, C, and D are shown in Table 2 below.

TABLE 2

| | Baseline | Subsequent | | |
|---|---|---|---|---|
| | morphology | morphology | Morphology | Threshold |
| Electrode | parameter | parameter | score | score |
| A | −12.0 millivolts | −12.1 mV | 1.0 | 0.6 |
| | (mV) | | | |
| B | −11.8 mV | −11.8 mV | 1.0 | 0.6 |
| C | −12.2 mV | −12.0 mV | 1.0 | 0.6 |
| D | −12.0 mV | −4.0 mV | 0.3 | 0.6 |

Corresponding threshold scores that are associated with the Electrodes A, B, C, and D also are displayed in the above table. The morphology scores are compared to the threshold scores to determine if the morphology scores indicate an initial cardiac event or a change in the progression of cardiac disease. For example, if the morphology score is less than the corresponding threshold score, then the morphology score may indicate an initial cardiac event or a worsening in the cardiac disease.

At 412, the differences between the morphology parameters are examined to determine if the differences indicate an initial cardiac event, such as an initial HF event or the onset of cardiac disease. In one embodiment, the differences between one or more subsequent morphology parameters and baseline parameters obtained by the distal Electrode D are examined to determine if the differences indicate an initial cardiac event. For example, the most distal electrode may be Electrode D that is located at or near the free wall of the left ventricle of the heart 106 (shown in FIG. 1). The electrophysiological responses sensed by Electrode D can provide the evoked response morphology of the free wall while the more proximal Electrodes A, B, and C provide the evoked response morphology of cardiac tissue that is closer to the left atrium of the heart 106.

During an initial cardiac event, such as an initial HF event, the free wall of the left ventricle may be subject to a greater increase in loading relative to the cardiac tissue of the left ventricle that is closer to the left atrium. As a result, the electrophysiological response sensed by Electrode D may change more and/or at an earlier time than the electrophysiological responses sensed by Electrodes A, B, or C. For example, as shown in FIG. 5, the electrophysiological responses sensed by the proximal Electrodes A, B, and C are approximately the same and do not significantly change between the baseline set 500 and the updated set 510 of electrophysiological responses.

With respect to the electrophysiological responses sensed by the distal Electrode D, however, the waveform of the subsequent electrophysiological response 518 of the updated set 510 is attenuated relative to the baseline electrophysiological response 508. For example, one or more of the positive peak value 314 (shown in FIG. 3), the absolute value of the negative peak value 312 (shown in FIG. 3), and/or the peak-to-peak height index 310 (shown in FIG. 3) may be decreased relative to the baseline electrophysiological response 508. Alternatively, the morphology of certain waveform segments of the subsequent electrophysiological response 518 may change relative to the baseline electrophysiological response 508. For example, the width index 322 (shown in FIG. 3) of the R-wave 306 (shown in FIG. 3) and/or the PDI 316 (shown in FIG. 3) in the subsequent electrophysiological response 518 may be greater than the width index 322 and/or PDI 316 of the baseline electrophysiological response 508. The significant differences between the subsequent and baseline morphologies 518, 508 of Electrode D while the less significant differences between the subsequent and baseline morphologies for the Electrodes A, B, and C may indicate an initial cardiac event, such as onset of cardiac disease or an initial HF event.

Alternatively, the differences between subsequent and baseline parameters obtained by one or more of the proximal Electrodes A, B, and/or C are examined to determine if the differences indicate an initial cardiac event. For example, one or more patients can have left ventricular anatomies that result in the electrophysiological responses sensed by the proximal Electrode A, B, and/or C to be changed more than the electrophysiological responses sensed by Electrode D during an initial cardiac event. As a result, instead of focusing the analysis on differences between electrophysiological responses sensed by the distal Electrode D, the analysis of whether an initial cardiac event is identified may focus on the differences between electrophysiological responses sensed by more proximal Electrodes A, B, and/or C.

In one embodiment, if the difference between the baseline and subsequent morphology parameters sensed by the distal Electrode D exceed an associated threshold, then the difference may indicate an initial cardiac event. As a result, flow of the method 400 flows from 412 to 414. With respect to the above example, the difference between the negative peak values 314 (shown in FIG. 3) of the baseline and subsequent morphology parameters of Electrode D exceed the associated threshold. Consequently, the difference indicates an initial cardiac event.

On the other hand, if the difference between the baseline and subsequent morphology parameters do not exceed the associated threshold, then the difference may not indicate an initial cardiac event. As a result, flow of the method 400 flows from 412 to 416. Alternatively, if an initial cardiac event previously was identified, flow of the method 400 may proceed from 412 to 416 regardless of the differences between the baseline and subsequent morphology parameters of the Electrode D.

At 414, an initial cardiac event is identified based on the difference between the baseline and subsequent morphology parameters of the distal Electrode D. For example, the onset of cardiac disease or an initial HF event may be identified. The identification of the initial cardiac event may be reported to an operator of the system 100 (shown in FIG. 1) by displaying a warning or alarm on the display device 110 (shown in FIG. 1). Flow of the method 400 may return to 404 where additional subsequent morphology parameters are monitored to track progression of the identified cardiac event or cardiac disease.

At 416, differences between the baseline and subsequent morphology parameters sensed at the monitoring locations of the heart 106 (shown in FIG. 1) are examined to identify progression of cardiac disease. The differences are examined to determine if the cardiac disease, such as HF, is becoming worse or is improving. After identifying onset of cardiac disease, such as an initial HF event, subsequent progression of the heart 106 into a more advanced or chronic cardiac disease may result in other regions of the heart 106 to experience significant increases in loading. These increases in loading can cause significant changes in the waveform morphology of electrophysiological responses sensed at the monitoring locations.

The differences between the baseline and subsequent morphology parameters at the monitoring locations can be compared to the associated thresholds to characterize changes in the progression of cardiac disease. In one embodiment, if at least a predetermined number of the differences sensed by the Electrodes A, B, C, and D exceed associated thresholds, then the progression of cardiac disease is identified as worsening or as not improving. For example, the loading of the left ventricle is still significantly increasing relative to a previous time. If the differences indicate that the progression of cardiac disease is worsening, then flow of the method 400 may proceed to 418.

On the other hand, if less than the predetermined number of differences exceeds associated thresholds, then the progression of cardiac disease may not be identified as worsening. For example, the progression may be identified as substantially unchanged or improving. As a result, flow of the method 400 may return to 404 where additional morphology parameters are obtained to continue to track the progression of cardiac disease.

At 418, the adverse progression of cardiac disease is identified. For example, the worsening of the cardiac disease may be visually reported to an operator of the system 100 (shown in FIG. 1) by displaying an alarm or warning on the display device 110 (shown in FIG. 1). Flow of the method 400 may return to 404 where additional morphology parameters are obtained to continue tracking the progression of cardiac disease.

Alternatively, the values of the morphology parameters and/or the morphology scores can be summed, multiplied, averaged, or otherwise combined and compared to a composite threshold. If the combined morphology parameters exceed a composite threshold and/or the combined morphology scores fall below a composite threshold score, then the progression of the cardiac disease may be identified as an adverse progression. Conversely, if the combined morphology parameters do not exceed a composite threshold and/or the combined morphology scores exceed a composite threshold score, then the progression of the cardiac disease may be identified as an improving progression.

With respect to the example embodiment of the electrophysiological responses shown in FIG. 5 and the differences recorded in the table 600 shown in FIG. 6, the differences between the morphology parameters for the Electrodes A, B, and C do not indicate that the cardiac disease is negatively progressing. As shown in FIG. 5, the subsequent electrophysiological responses 512, 514, 516 of the proximal Electrodes A, B, and C are not significantly attenuated relative to the corresponding baseline electrophysiological responses 502, 504, 506. As a result, the differences between the subsequent and baseline electrophysiological responses of the Electrodes A, B, and C do not indicate that the cardiac tissue at or near the corresponding monitoring locations is experiencing increased loading. This lack of increased loading can indicate that the cardiac disease is not worsening, is remaining substantially unchanged, or is improving. Consequently, flow of the method 400 returns to 404.

Upon returning to 404 of the method 400, additional stimulus pulses are applied to the heart 106 (shown in FIG. 1). At 406, an additional updated set 520 of electrophysiological responses 522, 524, 526, 528 is obtained by the Electrodes A, B, C, and D, as shown in FIG. 5. The updated set 520 may be obtained after the onset of cardiac disease or an initial HF event is identified at 414 of the method 400. The electrophysiological responses 522, 524, 526, 528 may be referred to as additional subsequent electrophysiological responses.

At 408, morphology parameters of the additional subsequent electrophysiological responses are determined, as described above. The morphology parameters may be referred to as additional subsequent morphology parameters. The additional subsequent morphology parameters can be recorded in the tables 600 (shown in FIG. 6). The second set of morphology parameters calculated after the baseline morphology parameters can be recorded in the third column 614 (shown in FIG. 6) of the tables 600 (shown in FIG. 6). The third column 614 is labeled “t₂” because the corresponding morphology parameters were obtained at a second time after the baseline morphology parameters and after the updated set 510 of electrophysiological responses were sensed.

At 410, the additional subsequent morphology parameters are compared to the baseline morphology parameters to identify changes or differences therebetween, as described above. As shown in FIG. 5, waveforms of the electrophysiological responses 522, 524, 526, 528 of the additional updated set 520 are attenuated relative to the electrophysiological responses of the updated set 510, which are attenuated relative to the electrophysiological responses of the baseline set 500. The additional attenuation of the electrophysiological responses over time may represent a worsening progression of cardiac disease, such as a worsening of HF.

If the differences between the additional subsequent morphology parameters and the baseline morphology parameters are significant, then the difference may indicate progression of cardiac disease. With respect to the negative peak value morphology parameters recorded in the table 600A of FIG. 6, differences between the negative peak values 312 (shown in FIG. 3) of the additional subsequent morphology parameters and the baseline morphology parameters are calculated. In the illustrated embodiment, the absolute values of the differences between the baseline and additional subsequent negative peak values 312 for the Electrodes A, B, C, and D are shown in Table 3 below.

TABLE 3

| | | Additional | | |
|---|---|---|---|---|
| | Baseline | subsequent | | |
| | morphology | morphology | Absolute | |
| Electrode | parameter | parameter | difference | Threshold |
| A | −12.0 millivolts | −7.2 mV | 4.8 mV | 4.0 mV |
| | (mV) | | | |
| B | −11.8 mV | −5.9 mV | 5.9 mV | 4.0 mV |
| C | −12.2 mV | −8.5 mV | 3.7 mV | 4.0 mV |
| D | −12.0 mV | −4.8 mV | 7.2 mV | 7.0 mV |

Alternatively, the differences between the baseline and additional subsequent morphology parameters may be represented by a morphology score, as described above. The morphology scores for the additional subsequent morphology parameters are shown in Table 4 below.

TABLE 4

| | | Additional | | |
|---|---|---|---|---|
| | Baseline | subsequent | | |
| | morphology | morphology | Morphology | Threshold |
| Electrode | parameter | parameter | score | score |
| A | −12.0 millivolts | −7.2 mV | 0.6 | 0.6 |
| | (mV) | | | |
| B | −11.8 mV | −5.9 mV | 0.5 | 0.6 |
| C | −12.2 mV | −8.5 mV | 0.7 | 0.6 |
| D | −12.0 mV | −4.8 mV | 0.5 | 0.6 |

The morphology scores may be compared to the threshold scores to determine if the morphology scores indicate a change in the progression of cardiac disease. For example, if the morphology score is less than the corresponding threshold score, then the morphology score may indicate an adverse progression of the cardiac disease.

In the illustrated embodiment, the differences between the baseline and the additional subsequent morphology parameters are significantly different from the baseline morphology parameters. As shown above in Tables 3 and 4, the morphology parameters of the additional updated set 520 of electrophysiological responses are significantly different from the baseline morphology parameters as the absolute differences between the morphology parameters exceed associated thresholds and/or the morphology scores are less than associated threshold scores. As a result, the differences between the morphology parameters indicate a potentially adverse progression of cardiac disease.

As described above, the initial cardiac event previously was identified at 412 and 414. Consequently, upon determining that significant differences exist between the morphology parameters at 410, flow of the method 400 may proceed to 416.

At 416, the differences between the baseline and the additional subsequent morphology parameters are examined to identify a change in the progression of cardiac disease. As described above, if at least a predetermined number of the differences between the baseline and the additional subsequent morphology parameters exceed associated thresholds, then the progression of cardiac disease is identified as worsening or as not improving. On the other hand, if less than the predetermined number of differences exceeds associated thresholds, then the progression of cardiac disease may not be identified as worsening.

In the example of the additional updated set 520 of electrophysiological responses shown in FIG. 5, three of the four differences or morphology scores indicate an adverse progression of the cardiac disease. For example, the differences associated with each Electrode A, B, C, and D exceed associated thresholds and the morphology scores associated with each Electrode A, B, C, and D are smaller than associated threshold scores. Therefore, flow of the method 400 proceeds to 418. Alternatively, if less than all of the differences and/or morphology scores indicate an adverse progression of cardiac disease (or less than a predetermined threshold of the differences and/or morphology scores indicate the adverse progression), the flow of the method 400 may return to 404, as described above.

At 418, the adverse progression of cardiac disease is identified. For example, the worsening of the cardiac disease may be visually reported to an operator of the system 100 (shown in FIG. 1) by displaying an alarm or warning on the display device 110 (shown in FIG. 1). In response to the adverse progression of the cardiac disease, a physician may change one or more aspects of a therapy or treatment provided to the patient. For example, the physician may change parameters of CRT therapy, medication administered to the patient, and/or the patient's diet.

As described above, flow of the method 400 may return to 404 where additional morphology parameters are obtained to continue tracking the progression of cardiac disease. For example, as shown in FIG. 5, another set 530 of electrophysiological responses 532, 534, 536, 538 may be obtained by the Electrodes A, B, C, and D. Morphology parameters of the electrophysiological responses 532, 534, 536, 538 are calculated and recorded in the fourth column 616 (shown in FIG. 6) of the tables 600 (shown in FIG. 6).

In one embodiment, after returning to 404 after an adverse progression of cardiac disease is identified, new or updated baseline morphology parameters may be obtained. For example, instead of continuing to compare newly sensed electrophysiological responses with the existing baseline morphology after an adverse progression of cardiac disease is identified, the existing baseline morphology parameters may be updated or recalculated as new or updated baseline morphology parameters. The updated baseline morphology parameters can represent the baseline morphology parameters during an identified cardiac disease state, or morphology parameters obtained after an adverse progression of cardiac disease has been identified. Similar to as described above, additional subsequent morphology parameters may be determined and compared to the updated baseline morphology parameters to track or monitor the progression of the previously identified cardiac disease.

The morphology parameters are then compared to the baseline morphology parameters to determine if the progression of the cardiac disease has changed. In the illustrated embodiment, the differences between each of the morphology parameters of the electrophysiological responses 532, 534 and the corresponding baseline morphology parameters may be relatively small or insignificant while the differences between each of the morphology parameters for the electrophysiological responses 536, 538 and the baseline morphology parameters may be relatively large or significant. As a result, two of the four morphology parameters of the set 520 of electrophysiological responses are significantly different from the baseline morphology parameters while three of the four morphology parameters of the previous set 510 were significantly different. Consequently, fewer morphology parameters deviate from the baseline morphology parameters. The decrease in number of morphology parameters that significantly deviate from the baseline morphology parameters may indicate an improving progression of cardiac disease, or that the cardiac disease is improving. This improvement may be communicated to the physician via the display device 110 (shown in FIG. 1) by a graphic or textual notice.

The progression of cardiac disease can be communicated to the operator of the system 100 (shown in FIG. 1), such as a physician, in a variety of ways. For example, the presence of HF and/or severity of HF can be assessed and communicated to the physician using the display device 110 (shown in FIG. 1). In one embodiment, the number of morphology parameters that exceed associated thresholds and/or the number of morphology scores that fall below associated threshold scores can be presented on the display device 110. Alternatively, the combined morphology parameters and/or the combined morphology scores can be presented on the display device 110.

The method 400 can proceed in a loop-wise manner to repeatedly examine morphology parameters of electrophysiological responses of the heart 106 (shown in FIG. 1) in several locations over time. The examinations of the morphology parameters may reveal trends in cardiac disease of the heart 106. For example, attenuation of the waveforms of the electrophysiological responses over time may indicate negatively progressing cardiac disease. Conversely, increasing amplitudes of the waveforms of the electrophysiological responses over time may indicate positively progressing cardiac disease, or response to therapy.

Additional or alternative sensors or measurements may be used to detect initial cardiac events and/or track the progression of cardiac disease. Various combinations of the electrodes of the IMD 102 (shown in FIG. 1) may measure cardiogenic impedance along vectors defined by the electrode combinations. For example, cardiogenic impedance vectors may be measured between the RV tip electrode 232 and each of the LV tip electrode 220, the LV ring electrode 222, the LV ring electrode 224, and the LV ring electrode 226 (all shown in FIG. 2). The cardiogenic impedance vectors may be one or more morphology parameters. Similar to as described above, baseline values for the cardiogenic impedance vectors may be obtained and compared to cardiogenic impedance vectors acquired at one or more later times. The differences between the baseline and subsequent vectors may be used to identify initial cardiac events and/or track the progress of cardiac disease similar to the manner in which baseline and subsequent morphology parameters are used.

As another example, waveform templates may be compared to one or more waveform segments of the electrophysiological responses sensed by the Electrodes A, B, C, and D. The waveform templates may comprise triangles or other shapes that approximate segments of the electrophysiological responses. Differences between the areas encompassed by the waveform templates and segments of the electrophysiological responses may be calculated as one or more of the morphology parameters described above. For example, the difference between the area of a waveform template and the PDI 316 (shown in FIG. 3) of an electrophysiological response may be calculated as a morphology parameter of the electrophysiological response.

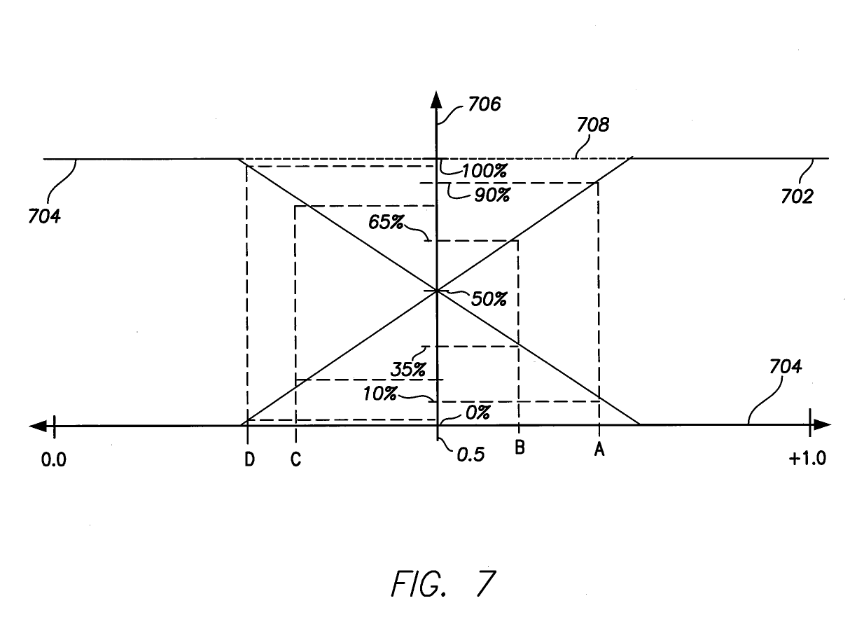

FIG. 7 illustrates probability curves 700, 702 that may be used in conjunction with the method 400 to determine the probability of an initial cardiac event and/or adverse progression of cardiac disease. The probability curves 700, 702 are shown alongside a horizontal axis 704 and a vertical axis 706. The horizontal axis 704 represents morphology scores of the morphology parameters and extends from 0.0 to +1.0. The vertical axis 706 represents percentages and extends from 0% to 100%. The horizontal axis 704 is disposed at 0% of the vertical axis 706 and a dashed line 708 is disposed at 100% of the vertical axis 706. The probability curve 700 is a non-HF probability curve that represents the probability of the heart 106 (shown in FIG. 1) not experiencing HF or another cardiac disease based on one or more morphology scores. The probability curve 702 is an HF probability curve that represents the probability of the heart 106 experiencing HF or another cardiac disease based on one or more of the morphology scores.

As described above, the morphology scores can represent the differences between morphology parameters of subsequent electrophysiological responses and the baseline morphology parameters. The morphology scores for the various Electrodes A, B, C, and D are positioned along the horizontal axis 704 according to the corresponding values of the morphology scores. In the illustrated embodiment, the following morphology scores are plotted along the horizontal axis 704:

TABLE 5

| Electrode | Morphology Score |
|---|---|
| A | 0.7 |
| B | 0.6 |
| C | 0.4 |
| D | 0.3 |

The morphology scores are shown in FIG. 7 using the letters of the corresponding Electrode A, B, C, or D. The probability curves 700, 702 are used to correlate a morphology score with a probability that the morphology score represents cardiac disease, such as an HF event. For example, the non-HF probability curve 700 for the morphology score of Electrode A indicates a 90% probability that the morphology score does not represent detected cardiac disease or a cardiac event while the HF probability curve 702 indicates a 10% probability that the morphology score does represent cardiac disease or a cardiac event. Similarly, the probability curves 700, 702 may provide probability of HF or non-HF events for the other morphology scores. In the illustrated embodiment, the following probabilities are determined based on the probability curves 700, 702:

| | | Non-HF | |
|---|---|---|---|
| Electrode | Morphology Score | Probability | HF Probability |
| A | 0.7 | 90% | 10% |
| B | 0.6 | 65% | 35% |
| C | 0.4 | 25% | 75% |
| D | 0.3 | 5% | 95% |

The HF and non-HF probabilities may be presented to a physician on the display device 110 (shown in FIG. 1). The physician may use the probabilities to determine how to provide or change treatment for the patient. For example, if only the distal Electrode D shows a high probability of cardiac disease, then the physician may determine that the cardiac disease is relatively new and/or representative of an initial cardiac event. Alternatively, if several of the morphology scores represent relatively high probabilities of cardiac disease, then the physician may determine that the cardiac disease has negatively progressed into an acute or chronic stage. The choice of treatment by the physician may depend on whether the cardiac disease is in an early onset stage or an acute or chronic stage.

FIG. 8 illustrates a block diagram of exemplary internal components of the IMD 102. The IMD 102 includes a housing 800, which in turn may further include a connector (not shown) having a plurality of inputs. The inputs may include one or more of an LV tip input terminal (V_{L} TIP) 802, an LA ring input terminal (A_{L} RING) 804, an LA coil input terminal (A_{L} COIL) 806, an AR tip input terminal (A_{R} TIP) 808, an RV ring input terminal (V_{R} RING) 810, an RV tip input terminal (V_{R} TIP) 812, an RV coil input terminal 814, and an SVC coil input terminal 816. A case input terminal 818 may be coupled with the housing 800. The input terminals may be electrically coupled with the electrodes shown in FIG. 2.

The IMD 102 includes a programmable microcontroller 820, which controls the operation of the IMD 102 based on acquired cardiac signals. The microcontroller 820 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. Among other things, the microcontroller 820 receives, processes, and manages storage of digitized data from the electrodes shown in FIG. 2. The microcontroller 820 may include one or more modules and processors configured to perform one or more of the actions and determinations described above in connection with the method 400 (shown in FIG. 4).

For example, an excitation module 822 determines when stimulus pulses are delivered to the heart 106 (shown in FIG. 1) in order to sense the electrophysiological responses of the heart 106. The excitation module 822 may direct the stimulus pulses to be supplied on a periodic basis.

A monitoring module 824 examines cardiac signals sensed by the electrodes shown in FIG. 2 and calculates the morphology parameters based thereon. For example, the monitoring module 824 may calculate the morphology parameters described above for the electrophysiological responses that are sensed by the electrodes. The monitoring module 824 communicates the morphology parameters to the memory 826 of the IMD 102. The monitoring module 824 retrieves baseline morphology parameters from the memory 826 and calculates differences between the baseline and subsequent morphology parameters, as described above. The differences may be calculated as absolute differences and/or morphology scores, also as described above. The monitoring module 824 communicates the morphology parameters, baseline morphology parameters, differences, and morphology scores to the memory 826 as progression data for storage in the memory 826.

A switch 828 includes several switches for connecting the electrodes shown in FIG. 2 and input terminals 802, 804, 806, 808, 810, 812, 814, 816 to the appropriate I/O circuits. The switch 828 closes and opens switches to provide electrically conductive paths between the circuitry of the IMD 102 and the input terminals in response to a control signal 830 from the microcontroller 820.

The cardiac signals sensed by the IMD 102 are applied to the inputs of an analog-to-digital (A/D) data acquisition system 866. The data acquisition system 866 is configured to acquire IEGM signals, convert the raw analog data into a digital IEGM signals, communicate the digital IEGM signals to the microcontroller 820, store the digital IEGM signals in the memory 826 for later processing, and/or communicate the signals to the external device 108. A control signal 868 from the microcontroller 820 determines when the data acquisition system 866 acquires signals, stores them in the memory 826, or transmits data to the microcontroller 820 and/or external device 108.

An impedance measuring circuit 832 may be electrically coupled to the switch 828 so that impedance vectors between combinations of the electrodes shown in FIG. 2 may be obtained. An atrial sensing circuit 834 and a ventricular sensing circuit 836 may be selectively coupled to the leads shown in FIG. 2 of the IMD 102 through the switch 828 for sensing the cardiac signals of the heart 106 (shown in FIG. 1), such as the electrophysiological responses. Control signals 838, 840 from the microcontroller 820 direct output of the atrial and ventricular sensing circuits 834, 836.

The memory 826 may be embodied in a tangible and/or non-transitory computer-readable storage medium such as a ROM, RAM, flash memory, or other type of memory. The microcontroller 820 is coupled to the memory 826 by a suitable data/address bus 842. The memory 826 may store programmable operating parameters and thresholds used by the microcontroller 820, as required, in order to customize the operation of IMD 102 to suit the needs of a particular patient. The memory 826 may store progression data, such as morphology parameters and morphology scores, as well as baseline morphology parameters, baseline morphology scores, thresholds, tables 600 (shown in FIG. 6), cardiac disease probabilities, and the like.

Data may be transmitted from and received by the IMD 102 through a telemetry circuit 844 in communication with the external device 108, such as a trans-telephonic transceiver or a diagnostic system analyzer. The telemetry circuit 844 is activated by a control signal 846 from the microcontroller 820. The telemetry circuit 844 allows progression data, such as morphology parameters and morphology scores, as well as baseline morphology parameters, baseline morphology scores, thresholds, tables 600 (shown in FIG. 6), cardiac disease probabilities, and the like, to be sent to the external device 108 through an established communication link 848.

The IMD 102 includes a battery 850 that provides operating power to the circuits shown within the housing 800, including the microcontroller 820. The IMD 102 also includes a physiologic sensor 852 that may be used to adjust pacing stimulation rate according to the exercise state of the patient.

In the case where IMD 102 is intended to operate as an ICD device, the IMD 102 detects the occurrence of a shift in one or more waveforms in detected cardiac signals that indicates an arrhythmia, and automatically applies an appropriate electrical shock therapy to the heart aimed at terminating the detected arrhythmia. To this end, the microcontroller 820 further controls a shocking circuit 854 by way of a control signal 856.

The IMD 102 includes an atrial pulse generator 858 and a ventricular pulse generator 860 to generate stimulation pulses, such as the stimulus pulses that are delivered to the heart 106 (shown in FIG. 1) to generate the electrophysiological responses. The pulse generators 858, 860 are controlled by the microcontroller 820 via appropriate control signals 862, 864 to trigger or inhibit the stimulation pulses.

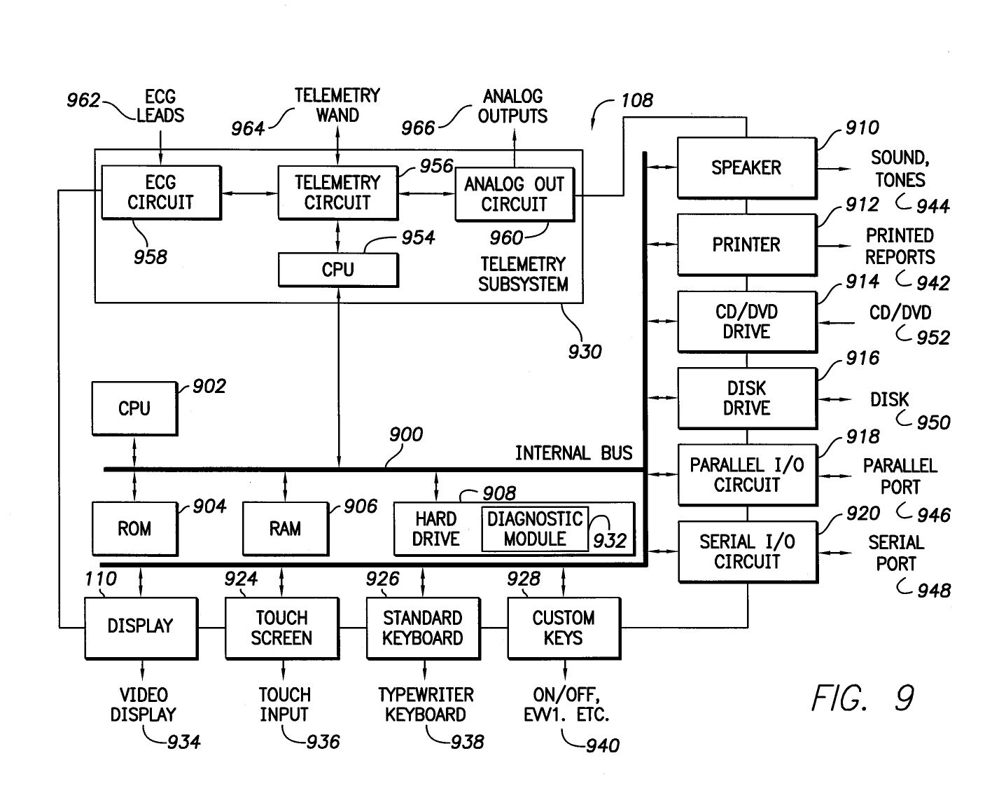

FIG. 9 illustrates a functional block diagram of the external device 108, such as a programmer, that is operated to interface with IMD 102. As described above, the external device 108 may be used by a physician or operator of the IMD 102 to monitor the detection of an initial cardiac event and/or the progression of cardiac disease, as well as monitor progression data such as morphology parameters, morphology scores, baseline morphology parameters, baseline morphology scores, thresholds, tables 600 (shown in FIG. 6), cardiac disease probabilities, and the like. The external device 108 may be used to program thresholds, threshold scores, baseline morphology parameters, and the like, into the IMD 102.

The external device 108 includes an internal bus 900 that connects/interfaces with a Central Processing Unit (CPU) 902, ROM 904, RAM 906, a hard drive 908, a speaker 910, a printer 912, a CD-ROM and/or DVD drive 914, an external disk drive 916, a parallel I/O circuit 918, a serial I/O circuit 920, the display device 110, a touch screen 924, a standard keyboard connection 926, custom keys 928, and a telemetry subsystem 930. The internal bus 900 is an address/data bus that transfers information between the various components described herein. The hard drive 908 may store progression data such as morphology parameters, morphology scores, baseline morphology parameters, baseline morphology scores, thresholds, tables 600 (shown in FIG. 6), cardiac disease probabilities, and the like.

The hard drive 908 may include a diagnostic module 932 that identifies changes in cardiac instability of the heart 106 (shown in FIG. 1) based on the progression data. For example, the diagnostic module 932 may identify an initial cardiac event and/or changes in the progression of cardiac disease based on the progression data, as described above in connection with the method 400 (shown in FIG. 4). Alternatively, the diagnostic module 932 may be provided in the microcontroller 820 (shown in FIG. 8) of the IMD 102.

The CPU 902 typically includes a microprocessor, a micro-controller, or equivalent control circuitry, designed specifically to control interfacing with the external device 108 and with the IMD 102. The CPU 902 may include RAM or ROM memory 904, logic and timing circuitry, state machine circuitry, and I/O circuitry to interface with the IMD 102. The display 110 (e.g., may be connected to the video display 934) and the touch screen 924 display graphic information relating to the IMD 102. The touch screen 924 accepts a user's touch input 936 when selections are made. The keyboard 926 (e.g., a typewriter keyboard 938) allows the user to enter data to the displayed fields, as well as interface with the telemetry subsystem 930. Furthermore, custom keys 928 turn on/off 940 the external device 108. The printer 912 prints copies of reports 942 for a physician to review or to be placed in a patient file, and speaker 910 provides an audible warning (e.g., sounds and tones 944) to the user. The parallel I/O circuit 918 interfaces with a parallel port 946. The serial I/O circuit 920 interfaces with a serial port 948. The disk drive 916 accepts disks 950. Optionally, the disk drive 916 may include a USB port or other interface capable of communicating with a USB device, such as a memory stick. The CD/DVD drive 914 accepts CDs and/or DVDs 952.

The telemetry subsystem 930 includes a central processing unit (CPU) 954 in electrical communication with a telemetry circuit 956, which communicates with both an ECG circuit 958 and an analog out circuit 960. The ECG circuit 958 is connected to ECG leads 962. The telemetry circuit 956 is connected to a telemetry wand 964. The analog out circuit 960 includes communication circuits to communicate with analog outputs 966. The external device 108 may wirelessly communicate with the IMD 102 and utilize protocols, such as Bluetooth, GSM, infrared wireless LANs, HIPERLAN, 3G, satellite, as well as circuit and packet data protocols, and the like. Alternatively, a hard-wired connection may be used to connect the external device 108 to the IMD 102.

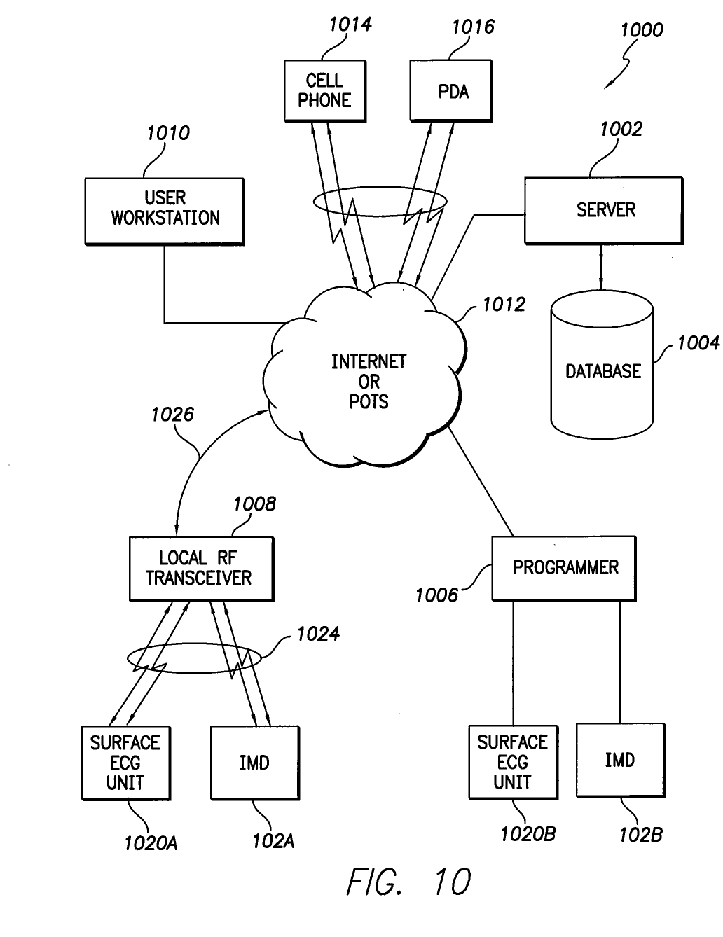

FIG. 10 illustrates a distributed processing system 1000 in accordance with one embodiment. The distributed processing system 1000 includes a server 1002 connected to a database 1004, a programmer 1006 (for example, similar to the external device 108 (shown in FIG. 1)), a local RF transceiver 1008, and a user workstation 1010 electrically connected to a communication system 1012.

The communication system 1012 may be the Internet, a voice over IP (VoIP) gateway, a local plain old telephone service (POTS) such as a public switched telephone network (PSTN), a cellular phone based network, and the like. Alternatively, the communication system 1012 may be a local area network (LAN), a campus area network (CAN), a metropolitan area network (MAN), or a wide area network (WAM). The communication system 1012 serves to provide a network that facilitates the transfer/receipt of progression data, such as morphology parameters and morphology scores, as well as baseline morphology parameters, baseline morphology scores, thresholds, tables 600 (shown in FIG. 6), cardiac disease probabilities, and the like.

The server 1002 is a computer system that provides services to other computing systems over a computer network. The server 1002 controls the communication of information such as the progression data. The server 1002 interfaces with the communication system 1012 to transfer information between the programmer 1006, the local RF transceiver 1008, the user workstation 1010, as well as a cell phone 1014 and a personal data assistant (PDA) 1016 to the database 1004 for storage/retrieval of progression data. On the other hand, the server 1002 may upload raw cardiac signals from a surface ECG unit 1020A, 1020B or an IMD 102A, 102B (such as the IMD 102) via the local RF transceiver 1008 or the programmer 1006.

The database 1004 stores the progression data for a single or multiple patients. The data is downloaded into the database 1004 via the server 1002 or, alternatively, the information is uploaded to the server 1002 from the database 1004. The programmer 1006 is similar to the external device 108 and may reside in a patient's home, a hospital, or a physician's office. Programmer 1006 interfaces with the surface ECG unit 1020B and the IMD 102B. The programmer 1006 may wirelessly communicate with the IMD 102B and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 1006 to the IMD 102B. The programmer 1006 is able to acquire cardiac signals from the surface of a person (e.g., ECGs), intra-cardiac electrogram (e.g., IEGM) signals from the IMD 102, and/or progression data, such as morphology parameters and morphology scores, as well as baseline morphology parameters, baseline morphology scores, thresholds, tables 600 (shown in FIG. 6), cardiac disease probabilities, and the like. The programmer 1006 interfaces with the communication system 1012, either via the Internet or via POTS, to upload the information acquired from the surface ECG unit 1020A, 1020B or the IMD 102A, 102B to the server 1002.

The local RF transceiver 1008 interfaces with the communication system 1012 via a communication link 1026, to upload progression data acquired from the surface ECG unit 1020A and/or the IMD 102A to the server 1002. In one embodiment, the surface ECG unit 1020A and the IMD 102A have a bi-directional connection with the local RF transceiver 1008 via a wireless connection 1024. The local RF transceiver 1008 is able to acquire cardiac signals from the surface of a person, intra-cardiac electrogram signals from the IMD 102A, and/or progression data from the IMD 102A. On the other hand, the local RF transceiver 1008 may download data, such as thresholds, threshold scores, baseline morphology parameters, and the like, to the surface ECG unit 1020A or the IMD 102A.

The user workstation 1010 may interface with the communication system 1012 via the internet or POTS to download progression data via the server 1002 from the database 1004. Alternatively, the user workstation 1010 may download raw data from the surface ECG unit 1020A, 1020B or IMD 102A, 102B via either the programmer 1006 or the local RF transceiver 1008. Once the user workstation 1010 has downloaded the progression data, the user workstation 1010 may process the information in accordance with one or more of the operations described above in connection with the method 400 (shown in FIG. 4). The user workstation 1010 may download the information and supply results of analyzing the progression data (such as cardiac disease probabilities) to the cell phone 1016, the PDA 1018, the local RF transceiver 1008, the programmer 1006, or to the server 1002 to be stored on the database 1004.

As used throughout the specification and claims, the phrases “computer-readable medium” and “instructions configured to” shall refer to any one or all of (i) computer-readable media or memory, software source code, software object code, hard wired logic, and/or software applications that direct processors, microprocessors, microcontrollers, and the like, to perform one or more directed operations.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the disclosed subject matter without departing from its scope. While the dimensions and types of materials described herein are intended to define the parameters of the described subject matter, they are by no means limiting and are exemplary embodiments. Many other embodiments will be apparent to those of skill in the art upon reviewing the above description. The scope of the claimed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the described subject matter, including making and using any devices or systems and performing any incorporated methods. The patentable scope of the disclosed subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of monitoring progression of cardiac disease, the method including:
applying stimulus pulses to a heart;
sensing cardiac signals of the heart at a plurality of different monitoring locations of the heart, the cardiac signals representative of electrophysiological responses of the heart to the stimulus pulses;
comparing a previously sensed electrophysiological response that is sensed near a first location of the monitoring locations with a subsequently sensed electrophysiological response that is sensed near the first location;
comparing a previously sensed electrophysiological response that is sensed near a second location of the monitoring locations with a subsequently sensed electrophysiological response that is sensed near the second location; and
identifying a change in progression of cardiac disease of the heart based on a difference between the previously and subsequently sensed electrophysiological responses at the first location and based on a difference between the previously and subsequently sensed electrophysiological responses at the second location.

2. The method of claim 1, wherein sensing cardiac signals comprises sensing the cardiac signals using a plurality of electrodes joined to a common lead assembly of a medical device, the cardiac signals used to determine the electrophysiological responses associated with different electrodes.

3. The method of claim 1, wherein identifying the change in progression of cardiac disease comprises detecting an initial event of cardiac disease based on the electrophysiological response sensed at a distal monitoring location of the monitoring locations, the change in progression of cardiac disease based on comparisons between the previously and subsequently sensed electrophysiological responses obtained at one or more proximal monitoring locations of the monitoring locations.

4. The method of claim 1, wherein identifying the change in progression of cardiac disease comprises identifying an adverse progression in the cardiac disease of the heart when a cardiac signal waveform segment of the subsequently sensed electrophysiological response obtained at one or more of the monitoring locations is attenuated relative to the previously sensed electrophysiological response obtained at the same one or more monitoring locations.

5. The method of claim 1, wherein identifying the change in progression of cardiac disease comprises identifying an improving progression in the cardiac disease of the heart when an amplitude of a first cardiac signal waveform segment of the subsequently sensed electrophysiological response obtained at one or more of the monitoring locations is larger than an amplitude of a second cardiac signal waveform segment of the previously sensed electrophysiological response obtained at the same one or more monitoring locations.

6. The method of claim 1, further comprising determining morphology parameters of cardiac signal waveform segments of the previously and subsequently sensed electrophysiological responses obtained at one or more of the monitoring locations and the comparing includes comparing the morphology parameters in order to identify the change in progression of the cardiac disease.

7. The method of claim 6, wherein the morphology parameters include at least one of negative peak indices, peak positive indices, peak-to-peak height indices, paced depolarization integrals (PDI), slope indices, or width indices of the cardiac signal waveform segments.

8. The method of claim 1, wherein sensing cardiac signals comprises sensing the previously sensed electrophysiological responses during a non-heart failure (HF) event to determine baseline morphology parameters and sensing the subsequently sensed electrophysiological responses during an HF event to determine subsequent morphology parameters.

9. The method of claim 8, wherein the comparing includes comparing the subsequent morphology parameters with the baseline morphology parameters and the identifying includes determining the change in progression based on one or more differences between the subsequent and baseline morphology parameters.

10. The method of claim 1, wherein sensing cardiac signals includes sensing the previously sensed electrophysiological response during a first time period that a patient having the heart is in a sedentary state and sensing the subsequently sensed electrophysiological response during a different, second time period that the patient is in a non-sedentary state.

11. A cardiac monitoring system comprising:
an implantable medical device configured to deliver stimulus pulses to a heart, the implantable medical device including electrodes configured to sense cardiac signals representative of electrophysiological responses of the heart to the stimulation pulses at a plurality of different monitoring locations of the heart;
a monitoring module configured to compare a previously sensed electrophysiological response that is sensed near a first location of the monitoring locations with a subsequently sensed electrophysiological response that is sensed near the first location, the monitoring module configured to compare a previously sensed electrophysiological response that is sensed near a second location of the monitoring locations with a subsequently sensed electrophysiological response that is sensed near the second location; and
a diagnostic module configured to identify a change in progression of cardiac disease of the heart based on a difference between the previously and subsequently sensed electrophysiological responses obtained near the first location and based on a difference between the previously and subsequently sensed electrophysiological responses obtained near the second location.

12. The system of claim 11, wherein the electrodes are joined to a common lead assembly of the implantable medical device.

13. The system of claim 11, wherein the diagnostic module identifies the change in progression of the cardiac disease based on differences between the electrophysiological responses and previously acquired baseline electrophysiological responses.

14. The system of claim 11, wherein the diagnostic module identifies an initial event of cardiac disease based on changes in the electrophysiological responses sensed at a distal monitoring location of the monitoring locations.

15. The system of claim 14, wherein the diagnostic module identifies the change in progression of the heart disease based on changes in the electrophysiological responses sensed at one or more proximal monitoring locations of the monitoring locations.

16. The system of claim 11, wherein the diagnostic module identifies an adverse progression of the cardiac disease when a cardiac signal waveform segment of the subsequently sensed electrophysiological response is attenuated relative to the previously sensed electrophysiological response obtained at the same monitoring location.

17. The system of claim 11, wherein the diagnostic module identifies an adverse progression of the cardiac disease when an amplitude of a cardiac signal waveform segment of the subsequently sensed electrophysiological response is larger than an amplitude of the previously sensed electrophysiological response obtained at the same monitoring location.

18. The system of claim 11, wherein the monitoring module calculates morphology parameters of cardiac signal waveform segments at one or more of the monitoring locations and the diagnostic module compares the morphology parameters in order to identify the change in progression of the cardiac disease.

19. The system of claim 18, wherein the morphology parameters include at least one of negative peak indices, peak positive indices, peak-to-peak height indices, paced depolarization integrals (PDI), slope indices, or width indices of the cardiac signal waveform segments.

20. The system of claim 11, wherein at least one of the monitoring module or the diagnostic module is disposed within a housing of the implantable medical device.

21. A tangible and non-transitory computer readable storage medium for use in a cardiac monitoring system including a processor and an implantable medical device that delivers stimulus pulses to a heart and senses cardiac signals of the heart in response thereto, the computer readable storage medium comprising instructions to direct the processor to:
determine electrophysiological responses of the heart to the stimulus pulses at a plurality of different monitoring locations of the heart;
compare a previously sensed electrophysiological response obtained near a first location of the monitoring locations with a subsequently sensed electrophysiological response obtained near the first location;
compare a previously sensed electrophysiological response obtained near a second location of the monitoring locations with a subsequently sensed electrophysiological response obtained near the second location; and
identify a change in progression of cardiac disease based on a difference between the previously and subsequently sensed electrophysiological responses obtained near the first location and based on a difference between the previously and subsequently sensed electrophysiological responses obtained near the second location.

22. The computer readable storage medium of claim 21, wherein the instructions direct the processor to:
determine a plurality of the electrophysiological responses of the heart that are associated with application of a plurality of stimulus pulses applied to the heart over a period of time; and
track changes in cardiac disease based on one or more trends in the electrophysiological responses over the period of time.

23. The computer readable storage medium of claim 21, wherein the instructions direct the processor to identify the change in progression of the cardiac disease based on differences between the electrophysiological responses and previously acquired baseline electrophysiological responses at each of the first and second locations.

24. The computer readable storage medium of claim 21, wherein the diagnostic module is configured to identify an adverse progression in the cardiac disease when a first cardiac signal waveform segment of the subsequently sensed electrophysiological response is attenuated relative to a second cardiac signal waveform segment of the previously sensed electrophysiological response at one or more of the monitoring locations.

25. The computer readable storage medium of claim 22, wherein the instructions direct the processor to compare the previous electrophysiological responses obtained during a non-heart failure (HF) event with the subsequent electrophysiological responses obtained during an HF event.
